(19) United States
(12) Patent Application Publication
(10) Pub. No.: US 2007/0278786 A1
(43) Pub. Date: Dec. 6, 2007

(54) Fluid Coupling System

(76) Inventors: Victor B. Mezhinsky, Brea, CA (US); G. Lamar Kirchhevel, Laguna Niguel, CA (US); Paul D. Zabilski, Tustin, CA (US)

Correspondence Address: ALCON, IP LEGAL, TB4-8, 6201 SOUTH FREEWAY, FORT WORTH, TX, 76134, US

(21) Appl. No.: 11/767,068

(22) Filed: Jun. 22, 2007

Related U.S. Application Data
(63) Continuation of application No. PCT/US07/65600, filed on Mar. 30, 2007.
Application No. PCT/US07/65600 is a continuation of application No. 11/418,605, filed on May 5, 2006, now abandoned.

(51) Int. Cl. F16L 37/084

(52) U.S. Cl. 285/39; 285/81

(57) ABSTRACT

A fluid coupling with a rotating outer collar includes keys or tabs which engage keyways or slots to prevent rotation of the outer collar.

This application is a continuation of PCT/US2007/065600 filed Mar. 30, 2007 entitled “Fluid Coupling System,” which claims priority from U.S. application Ser. No. 11/418,605 filed May 5, 2006.

### FIELD

This invention pertains to a coupling system to connect flow lines used to guide the flow of liquid, air or other gases; more particularly, the disclosed coupling system pertains to a pneumatic coupling system used for the connection of either a surgical hand piece or a probe through which pressurized air or gas flows to the control console portion of a piece of medical/surgical equipment.

### BACKGROUND

While the disclosed invention is described in terms of a pneumatic coupling system for use on a vitrectomy system, those of ordinary skill in the art will understand that the utility of the disclosed invention covers those applications where a quick connect and disconnect coupling is used on any gas or liquid flow line associated with a control console.

There are a wide variety of prior art quick connect and disconnect pneumatic couplings presently available; e.g., those manufactured by the Cole-Palmer Instrument Company and those manufactured by Colder Products. Many of the currently available quick connect and disconnect pneumatic couplings include a rotating outer sleeve.

There are several patents which describe quick connect and disconnect pneumatic couplings similar to the present invention; however, the prior art coupling systems described in the paragraphs below are complex and not suitable for use in a medical/surgical system such as a vitrectomy system.

U.S. Pat. No. 6,443,496 B2 describes a connector for a fluid handling system. The male connector retaining element is a body flange, and the female connector retaining element includes a pair of diametrically opposed retainers.

U.S. Pat. No. 6,264,248 B1 describes a quick coupling system for use with a hose. The hose end, having a clamping ring, actually screws onto the mating coupling, thus ensuring a tight connection.

U.S. Pat. No. 5,799,987 describes a fluid fitting coupling system. The female fitting assembly has a retaining lip that protrudes inwardly and the male fitting assembly has a flange that protrudes outwardly. By engaging both the male and female fittings and twisting one about a central axis, the disclosed coupling system provides tight union between the hose ends.

In some applications the presence of a rotating outer sleeve on a quick connect and disconnect pneumatic coupling has proven to be detrimental. To solve the problem of eliminating the rotating outer sleeve from a quick connect and disconnect pneumatic coupling, one solution has been to add a pressed-in console dowel pin to engage the rotating sleeve. The pressed-in console dowel pin mechanically prevents the sleeve from spinning around the quick connect and disconnect pneumatic coupling. The use of a pressed-in console dowel pin does provide an advantage in that it enables the user to connect the tubing connector (body part), which is a consumable product, to a console coupling insert with one hand. While the use of a pressed-in console dowel pin has provided satisfactory results, it has been found that the use of a console dowel pin has the significant drawback of interfering with the Radio Frequency Identification (RFID) of the quick connect and disconnect pneumatic coupling.

There remains a need in the art to provide a quick connect and disconnect pneumatic coupling with a non-rotatable outer sleeve. Further, there remains a need in the art to eliminate the console dowel pin to remove any source of the interference with the RFID of the quick connect and disconnect pneumatic coupling.

### SUMMARY

The disclosed fluid coupling system provides a non-rotating outer sleeve that eliminates the disadvantages of conventional coupling systems.

In the insert portion of the disclosed fluid in-line quick connect and disconnect fluid coupling there is an outer sleeve that has a latching feature which prevents the outer portion of the fluid coupling from spinning about its central axis. The design of this latching feature has two configurations. The first configuration includes one or more keys on the outer sleeve which engage with a slot in a mating body part. The second configuration includes one or more tabs on the outer sleeve which engage with a back mounting plate or a fluid manifold.

### BRIEF DESCRIPTION OF THE DRAWING FIGURES

A better understanding of the fluid coupling system of the present invention may be had by reference to the drawing figures wherein:

FIG. 1 is a perspective view of the keyed sleeve version of the fluid coupling system of the present invention;

FIG. 2 is a side elevational view in partial section of the tabbed sleeve version of the fluid coupling system connected to a back mounting plate or a fluid manifold; and

FIG. 3 is a perspective view of the tabbed sleeve version of the fluid coupling system shown in FIG. 2.

### DESCRIPTION OF THE EMBODIMENTS

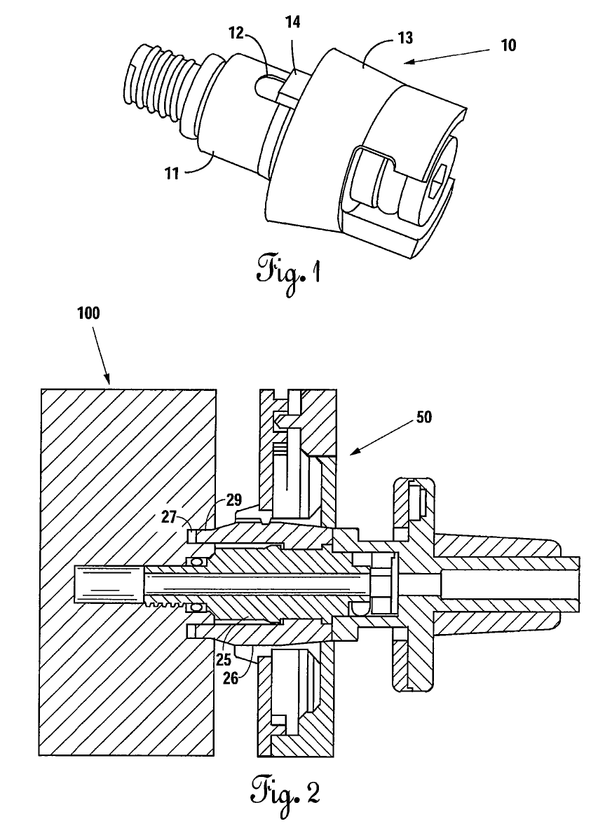

FIG. 1 depicts the engagement of a key 14 on the outer rotating sleeve 13 with a slot 12 or keyway formed in the central male body portion of the quick connect and disconnect coupling 10 of the present invention. The rotating outer sleeve 13 portion of the quick connect and disconnect coupling 10 includes one or more opposing keys 14. The central male body portion 11 has one or more slots or keyways 12. During the assembly of the coupling 10, a key 14 passes into a slot 12, thus providing a positive engagement. Because of the engagement of the keys 14 with the slots 12, the rotating outer sleeve 13 will not rotate around the coupling 10.

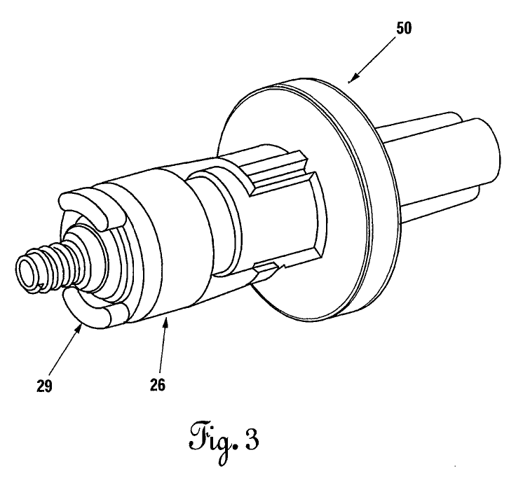

FIGS. 2 and 3 depict the tabbed sleeve version 50 of the disclosed invention. In this embodiment 50, the rotating outer sleeve 26 has a set of at least one tab 29 extending therefrom. The mounting block of a pneumatic manifold 100 has a set of at least one flat surface arcuate slot 27. During the assembly, each tab 29 inserts into an arcuate slot 27, thus providing a positive engagement between the coupling 50 and the pneumatic manifold 100.

FIG. 3 is an perspective view of tabbed sleeve version 50 of the coupling. The engagement of the tabs 29 with the arcuate slots 27 in the pneumatic manifold 100 prevents the rotating sleeve 26 from turning about the central axis of the quick connect and disconnect coupling 50.

While the present invention has been described in terms of a key and keyway embodiment or a tab and slot embodiment, those of ordinary skill in the art will understand that other embodiments have been enabled by the foregoing disclosure. Such other embodiments shall fall within the scope and meaning of the appended claims.

What is claimed is:

1. A fluid coupling system for use with the console portion of a medical/surgical system, said fluid coupling comprising:
a central male body portion, said central male body portion including at least one slot;
a sleeve constructed and arranged to surround and rotate about said central male body portion, said sleeve including at least one key;
whereby upon assembly said key positively engages said slot to prevent the rotation of said sleeve.

2. A fluid coupling system for use with the console portion of a medical/surgical system, said fluid coupling system comprising:
a mounting block, said mounting block including a set of opposed arcuate slots;
a rotatable sleeve constructed and arranged to engage said mounting block, said rotatable sleeve including a set of opposed arcuate tabs;
whereby upon assembly said set of opposed tabs on said rotatable sleeve positively engage said set of opposed arcuate slots on said mounting block to prevent the rotation of said rotatable sleeve.

3. A method of preventing the rotation of the outer sleeve portion of a quick connect and disconnect fluid coupling, said method comprising the steps of:
forming at least one slot in the central male body portion of the quick connect and disconnect fluid coupling;
forming at least one key on the sleeve portion of the quick connect and disconnect fluid coupling;
whereby said sleeve portion will not rotate around said central male body portion because of the engagement of said key and said slot.

4. A method of preventing the rotation of the outer sleeve portion of a quick connect and disconnect fluid coupling constructed for engagement with a fluid manifold, said method comprising the steps of:
forming at least one arcuate slot in the fluid manifold;
forming at least one arcuate tab on the outer sleeve of the quick connect and disconnect fluid coupling;
whereby said outer sleeve of the quick connect and disconnect fluid coupling will not rotate when said at least one arcuate tab engages said at least one arcuate slot.
